(19) United States
(12) Patent Application Publication
(10) Pub. No.: US 2024/0367146 A1
(43) Pub. Date: Nov. 7, 2024

(54) HUMIDITY CONTROL AGENT AND DEVICE

(71) Applicant: DAIKIN INDUSTRIES, LTD., Osaka (JP)

(72) Inventors: Toshiyuki ITOH, Nagakute-shi (JP); Ryo Inoue, Osaka (JP); Tatsuya Ando, Osaka (JP); Shuji Ikegami, Osaka (JP)

(73) Assignee: DAIKIN INDUSTRIES, LTD., Osaka (JP)

(21) Appl. No.: 18/772,392

(22) Filed: Jul. 15, 2024

Related U.S. Application Data
(63) Continuation of application No. PCT/JP2023/001199, filed on Jan. 17, 2023.

(30) Foreign Application Priority Data
Jan. 19, 2022 (JP) 2022-006519

(51) Int. Cl. B01J 20/22; B01D 53/26

(52) U.S. Cl. CPC B01J 20/22; B01D 53/263; B01D 2252/2053

(57) ABSTRACT

A moisture control material comprises a salt composed of a specific triazolium cation and a phosphate ester anion.

### CROSS-REFERENCE TO RELATED APPLICATIONS

This application is a Rule 53 (b) Continuation of International Application No. PCT/JP2023/001199 filed Jan. 17, 2023, which claims priority based on Japanese Patent Application No. 2022-006519 filed Jan. 19, 2022, the respective disclosures of which are incorporated herein by reference in their entirety.

### TECHNICAL FIELD

The present disclosure relates to a moisture control material for use in an air conditioner and an absorption refrigerator, and an apparatus such as an air conditioner and an absorption refrigerator.

### BACKGROUND ART

A desiccant-type air conditioner uses a liquid moisture control material having properties of absorbing water vapor in the air. For example, in Non Patent Documents 1 and 2, liquid moisture control materials made of a lithium chloride aqueous solution, a calcium chloride aqueous solution, or triethylene glycol are disclosed. In Patent Documents 1 to 5 and Non Patent Documents 3 to 7, liquid moisture control materials made of ionic liquid are disclosed, and as the ionic liquid, salts of bromide anions and tetrafluoroborate, dimethyl phosphate anions or methylsulfate anions with imidazolium cations, alkyl phosphonium cations or quaternary ammonium cations are disclosed. In Non Patent Documents 8 and 9, salts of dimethyl phosphate anions with cholinium cations, dicationic and cyclic quaternary ammonium cations are disclosed.

### RELATED ART

### Patent Document

Patent Document 1: Japanese Translation of PCT International Application Publication No. 2014-505586
Patent Document 2: Japanese Patent Laid-Open No. 2017-221940
Patent Document 3: Japanese Patent Laid-Open No. 2017-154076
Patent Document 4: Japanese patent Laid-Open No. 2016-052614

### Non Patent Documents

Non Patent Document 1: L. Mei, Y. I. Dai, A technical review on use of liquid-desiccant dehumidification for air-conditioning application, Renewable and Sustainable Energy Reviews, 2008, 12, 662-689.
Non Patent Document 2: R. P. Singh, V. K. Mishra, R. K. Das, Desiccant materials for air condition in applications: A review, lop Conference Series. Materials Science and Engineering, 404 (2018), 012005.
Non Patent Document 3: L. E. Ficke, J. F. Brennecke, Interactions of Ionic Liquids and Water, J. Phy. Chem. B 114 (2010) 10496-10501.
Non Patent Document 4: L. Jing, Z. Danxing, F. Lihua, W. Xianghong, D. Li, Vapor Pressure Measurement of the Ternary Systems H₂O+LiBr+[Dmim]Cl, H₂O+LiBr+[Dmim]BF₄, H₂O+LiCl+[Dmim]Cl, and H₂O+LiCl+[Dmim]BF₄, J. Chem. Eng. Data 56 (2011) 97-101.
Non Patent Document 5: Y. Luo, S. Shao, H. Xu, C. Tian, Dehumidification performance of [EMIM]BF₄, Appl. Thermal Eng. 31 (2011) 2722-2777.
Non Patent Document 6: Y. Luo, S. Shao, F. Qin, C. Tian, H. Yang, Investigation on feasibility of ionic liquids used in solar liquid desiccant air conditioning system, Solar Energy 86 (2012) 2718-2724.
Non Patent Document 7: Watanabe, H.; Komura, T.; Matsumoto, R.; Ito, K.; Nakayama, H.; Nokami, T.; Itoh, T. Design of Ionic Liquids as Liquid Desiccant for an Air Conditioning System, Green Energy & Environment, 4 (2019), 139-145.
Non Patent Document 8: Maekawa, S.; Matsumoto, R.; Ito, K.; Nokami, T.; Li, J-X.; Nakayama, H.; Itoh, T.* Design of Quaternary Ammonium Type-Ionic Liquids as Desiccants for an Air-Conditioning System, Green Chemical Engineering, 1 (2020), 109-116.
Non Patent Document 9: Dicationic Type Quaternary Ammonium Salts as Candidates of Desiccants for an Air-Conditioning System, Itoh, T.; Hiramatsu, M.; Kamada, K.; Nokami, T.; Nakayama, H.; Yagi, K.; Yan, F.; Kim, H-J. ACS Sustainable Chem. Eng., 9 (2021), 14502-14514.

### SUMMARY

The present disclosure provides a moisture control material comprising a salt composed of a triazolium cation represented by the following formula (N1) and a phosphate ester anion:

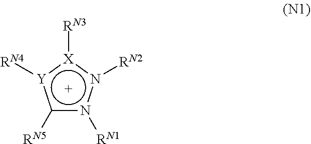

wherein in formula (N1), X and Y represent a nitrogen atom or a methine group, with proviso that any one of X and Y is a nitrogen atom, and another one is a methine group; R^{N1}, R^{N2}, R^{N3}, R^{N4} and R^{N5} are each independently absent, a hydrogen atom, an alkyl group having 1 to 10 carbon atoms which optionally has a hydroxy group and optionally includes one or more oxygen atoms in the chain; R^{N1}, R^{N2}, R^{N3}, R^{N4} and R^{N5} are the same or different from each other; not all of R^{N1}, R^{N2}, R^{N3}, R^{N4} and R^{N5} are hydrogen atoms or absent; and one or more of R^{N1}, R^{N2}, R^{N3} and R^{N4} substituted on a nitrogen atom that constitutes a 5-membered ring are alkyl groups having 1 to 10 carbon atoms, which optionally have a hydroxy group and optionally include one or more oxygen atoms in the chain.

### Effects

According to the present disclosure, a moisture control material having high moisture absorption properties, of which aqueous solution has low viscosity with low corrosiveness to metals, can be provided.

### BRIEF DESCRIPTION OF DRAWINGS

FIG. 1 is a graph showing measurement results of the water vapor absorption capacity of a specific salt (11).

FIG. 2 is a graph showing measurement test results of the equilibrium water vapor pressure of a specific salt (11).

FIG. 3 is a graph showing measurement test results of the corrosiveness to metals of a specific salt (11).

FIG. 4 is a graph showing measurement results of temperature-variable viscosity of each sample.

FIG. 5 is a graph showing ΔVP₅₀₋₂₅ for each sample.

### DESCRIPTION OF EMBODIMENTS

The lithium chloride aqueous solution and the calcium chloride aqueous solution disclosed in Non Patent Documents 1 to 2 have an advantage of being able to obtain low-humidity air in a stable state. However, in general, aqueous solutions of alkali metal salts and alkaline earth metal salts of these halide ions are corrosive to metals. Therefore, when these substances are applied to a moisture control material, there exists a problem that a highly corrosion-resistant material such as titanium is required for use in a part in contact with the moisture control material in apparatuses such as air conditioners and absorption refrigerators.

Although an ionic liquid composed of cholinium cations and lactic anions having high moisture absorption property is reported in Patent Document 1, lactic anions are unstable and cannot withstand long-term use.

The moisture control materials disclosed in Patent Documents 2 to 4 and Non Patent Documents 4 to 5 includes an ionic liquid composed of imidazolium cations and bromide anions and tetrafluoroborate anions as constituent anions. The bromide anions and the tetrafluoroborate anions have problems of difficult handleability in addition to corrosiveness to metals.

In Non Patent Document 3, it is reported that moisture absorption property of the ionic liquid of imidazolium salt mainly depends on anions, and that acetate anions have good water absorption properties. However, only imidazolium cations have been studied as cations, and an aqueous solution of imidazolium ionic liquid is highly corrosive to metals such as copper (Non Patent Document 7). It is also known that ionic liquids of acetate anions are unstable and cannot withstand long-term use (Non Patent Document 7). In order to solve such problems, in Non Patent Documents 7 to 9, dimethyl phosphate and methyl sulfate are given attention as anions for the selection of ionic liquids. In Non Patent Document 7, it is clarified that an ionic liquid including a combination of phosphonium cations and dimethyl phosphate anions has excellent moisture absorption properties, and in Non Patent Document 8, it is reported that an ionic liquid aqueous solution including a combination of cholinium cations and dimethyl phosphate anions exhibits high moisture absorption properties with low corrosiveness to metals. Further, in Non Patent Document 9, it is reported that quaternary ammonium=dimethyl phosphate salt, in particular, dicationic ammonium, exhibits higher moisture absorption properties in comparison with conventionally reported moisture control materials. However, it has been found that the aqueous solution of dicationic ammonium=dimethyl phosphate salt has a relatively high viscosity (Non Patent Document 9).

The present disclosure has been made in the light of the circumstances, and the object thereof is to provide a moisture control material that exhibits good moisture absorption properties (moisture absorption capacity and water vapor exchange capacity) and low viscosity, having low corrosiveness to metals.

Based on the above problems, the present inventors have conducted studies and found that the above problems can be solved by using a moisture control material containing a salt composed of triazolium cations and phosphate ester anions, having a specific structure.

Specifically, the problem has been solved by the following means.

<1> A moisture control material comprising a salt composed of a triazolium cation represented by the following formula (N1) and a phosphate ester anion:

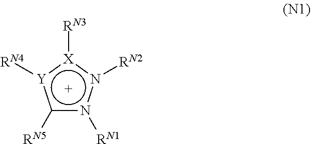

wherein in formula (N1), X and Y represent a nitrogen atom or a methine group, with proviso that any one of X and Y is a nitrogen atom, and another one is a methine group; R^{N1}, R^{N2}, R^{N3}, R^{N4} and R^{N5} are each independently absent, a hydrogen atom, an alkyl group having 1 to 10 carbon atoms which optionally has a hydroxy group and optionally includes one or more oxygen atoms in the chain; R^{N1}, R^{N2}, R^{N3}, R^{N4} and R^{N5} are the same or different from each other; not all of R^{N1}, R^{N2}, R^{N3}, R^{N4} and R^{N5} are hydrogen atoms or absent; and one or more of R^{N1}, R^{N2}, R^{N3} and R^{N4} substituted on a nitrogen atom that constitutes a 5-membered ring are alkyl groups having 1 to 10 carbon atoms, which optionally have a hydroxy group and optionally include one or more oxygen atoms in the chain.

<2> The moisture control material according to item <1>, wherein in formula (N1), in the case where X is a nitrogen atom, R^{N1} and R^{N3} are alkyl groups having 1 to 10 carbon atoms, which optionally have a hydroxy group and optionally include one or more oxygen atoms in the chain, and R^{N2} is absent.

<3> The moisture control material according to item <1>, wherein in formula (N1), in the case where Y is a nitrogen atom, R^{N1} and R^{N2} are alkyl groups having 1 to 10 carbon atoms, which optionally have a hydroxy group and optionally include one or more oxygen atoms in the chain, and R^{N4} is absent; alternatively, in the case where Y is a nitrogen atom, R^{N1} and R^{N4} are alkyl groups having 1 to 10 carbon atoms, which optionally have a hydroxy group and optionally include one or more oxygen atoms in the chain, and R^{N2} is absent.

<4> The moisture control material according to any one of items <1> to <3>, comprising a salt composed of triazolium cations represented by any one of the following formulas (1-1) to (1-3) and phosphate ester anions:

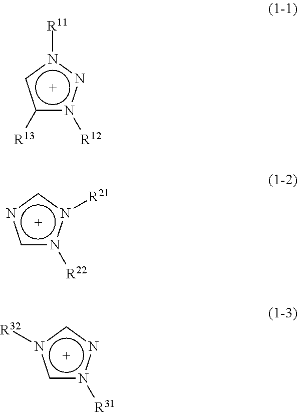

wherein in formula (1-1), R¹¹ and R¹² are alkyl groups having 1 to 10 carbon atoms which optionally have a hydroxy group and optionally include one or more oxygen atoms in the chain; R¹³ is a hydrogen atom or an alkyl group having 1 to 10 carbon atoms which optionally have a hydroxy group and optionally include one or more oxygen atoms in the chain; R¹¹, R¹² and R¹³ are the same or different from each other;
in formula (1-2), R²¹ and R²² are alkyl groups having 1 to 10 carbon atoms, which optionally have a hydroxy group and optionally include one or more oxygen atoms in the chain; R²¹ and R²² are the same or different from each other; and
in formula (1-3), R³¹ and R³² are alkyl groups having 1 to 10 carbon atoms, which optionally have a hydroxy group and optionally include one or more oxygen atoms in the chain; R³¹ and R³² are the same or different from each other.

<5> The moisture control material according to any one of items <1> to <4>, wherein the phosphate ester anion comprises an anion represented by formula (N2).

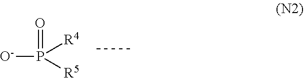

wherein in formula (N2), R⁴ is a hydrogen atom, a hydroxyl group, an alkyl group having 1 to 6 carbon atoms, a hydroxyalkyl group having 1 to 6 carbon atoms, an alkoxy group having 1 to 6 carbon atoms, an alkenyl group having 2 to 6 carbon atoms, a poly(alkyleneoxy) group having 10 or less carbon atoms, an alkylthio group having 1 to 6 carbon atoms, or a poly(alkylene thio) group having 10 or less carbon atoms; and R⁵ is a hydrogen atom, an alkyl group having 1 to 6 carbon atoms, an alkoxy group having 1 to 6 carbon atoms, an alkenyl group having 2 to 6 carbon atoms, a poly(alkyleneoxy) group having 10 or less carbon atoms, an alkylthio group having 1 to 6 carbon atoms, or a poly(alkylene thio) group having 10 or less carbon atoms.

<6> The moisture control material according to any one of items <1> to <5>, wherein the triazolium cation is represented by any of the following formulas (1) to (8), (34) and (35):

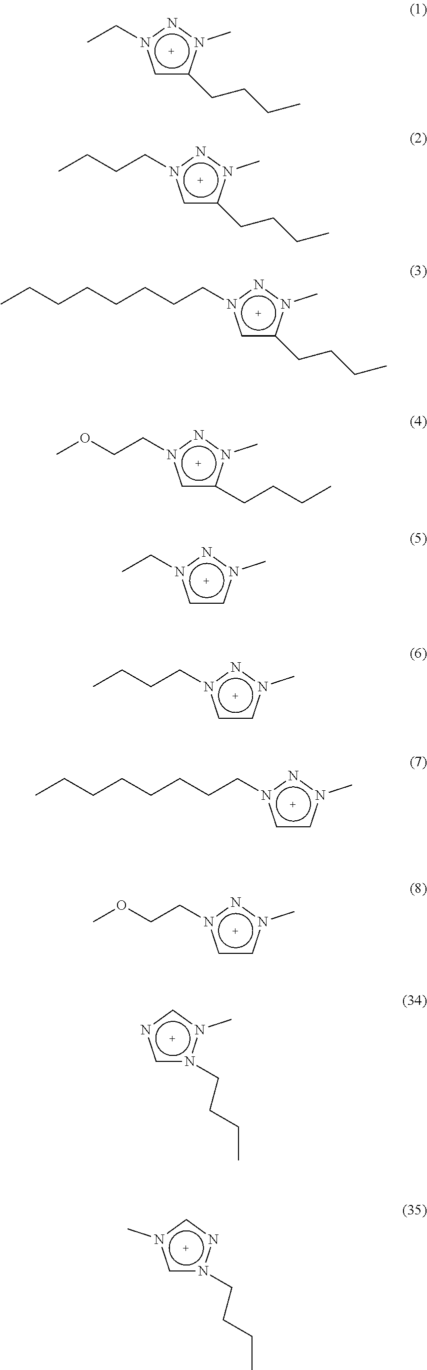

<7> The moisture control material according to any one of items <1> to <6>, wherein the phosphate ester anion is represented by the following formula (9) or (10):

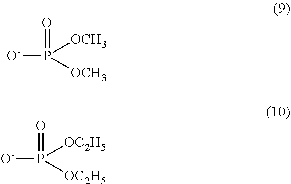 [SMILES: COP(=O)([O-])OC ; CCOP(=O)([O-])OCC]

<8> The moisture control material according to any one of items <1> to <7>, wherein a solution of the salt composed of triazolium cations and phosphate ester anions has a viscosity of 50 mPa·s or less at 25° C.

<9> The moisture control material according to any one of items <1> to <8>, having an equilibrium water vapor pressure at 25° C. of 100 hPa or less, and an equilibrium water vapor pressure at 50° C. of 150 hPa or more.

<10> The moisture control material according to any one of items <1> to <9>, having a value as difference between the equilibrium water vapor pressure at 25° C. and the equilibrium water vapor pressure at 50° C. (ΔVP₅₀₋₂₅) of 100 hPa or more.

<11> An apparatus comprising the moisture control material according to any one of items <1> to <10>.

An embodiment of the present dislosure is described in detail as follows. Incidentally, the following present embodiment is an example for illustrating the present dislosure, and the present dislosure is not limited to the present embodiment alone.

Incidentally, in the present specification, “to” is used to mean including the numerical values pre- and post-described as a lower limit and an upper limit.

In the present specification, various physical values and characteristic values are ones at 23° C. unless otherwise described.

In the notation of a group (atomic group) in the present specification, a notation without description of substitution or unsubstitution includes a group (atomic group) having no substituent and a group having a substituent (atomic group). For example, “alkyl group” includes not only an alkyl group having no substituent (unsubstituted alkyl group) but also an alkyl group having a substituent (substituted alkyl group). In the present specification, a notation without description of substitution or unsubstitution preferably means unsubstitution.

In the case where a standard shown in the present specification has a different measurement method or the like depending on years, the method or the like is based on the standard as of Jan. 1, 2022 unless otherwise described.

A moisture control material in the present embodiment comprises a salt composed of a triazolium cation represented by the following formula (N1) and a phosphate ester anion (hereinafter, described as a specific salt).

### <Triazolium Cation Represented by Formula (N1)>

A triazolium cation represented by the following formula (N1) for use in the present embodiment is described as follows.

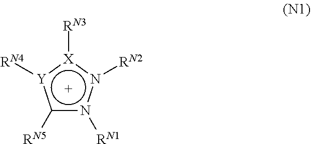

In formula (N1), X and Y represent a nitrogen atom or a methine group, with proviso that any one of X and Y is a nitrogen atom, and another one is a methine group; R^{N1}, R^{N2}, R^{N3}, R^{N4} and R^{N5} are each independently absent, a hydrogen atom, an alkyl group having 1 to 10 carbon atoms which optionally has a hydroxy group and optionally include one or more oxygen atoms in the chain; R^{N1}, R^{N2}, R^{N3}, R^{N4} and R^{N5} are the same or different from each other; not all of R^{N1}, R^{N2}, R^{N3}, R^{N4} and R^{N5} are hydrogen atoms or absent; one or more of R^{N1}, R^{N2}, R^{N3} and R^{N4} substituted on a nitrogen atom that constitutes a 5-membered ring are alkyl groups having 1 to 10 carbon atoms, which optionally have a hydroxy group and optionally include one or more oxygen atoms in the chain.

X and Y represent a nitrogen atom or a methine group, with proviso that any one of X and Y is a nitrogen atom, and another one is a methine group. In other words, both of X and Y are not nitrogen atoms, and both of them are not methine groups.

R^{N1}, R^{N2}, R^{N3}, R^{N4} and R^{N5} are each independently absent, a hydrogen atom, an alkyl group having 1 to 10 carbon atoms which optionally has a hydroxy group and optionally includes one or more oxygen atoms in the chain (hereinafter, the group may be referred to as specific alkyl group in some cases). R^{N1}, R^{N2}, R^{N3}, R^{N4} and R^{N5} are the same or different from each other. Not all of R^{N1}, R^{N2}, R^{N3}, R^{N4} and R^{N5} are hydrogen atoms or absent. One or more of R^{N1}, R^{N2}, R^{N3} and R^{N4} substituted on a nitrogen atom that constitutes a 5-membered ring are specific alkyl groups. It is particularly preferable that two of R^{N1}, R^{N2}, R^{N3} and R^{N4} substituted on a nitrogen atom be specific alkyl groups.

In formula (N1), in the case where X is a nitrogen atom, R^{N1} and R^{N3} are alkyl groups having 1 to 10 carbon atoms which optionally have a hydroxy group and optionally include one or more oxygen atoms in the chain, and R^{N2} is preferably absent.

In formula (N1), in the case where Y is a nitrogen atom, R^{N1} and R^{N2} are alkyl groups having 1 to 10 carbon atoms which optionally have a hydroxy group and optionally include one or more oxygen atoms in the chain, and R^{N4} is absent; alternatively, in the case where Y is a nitrogen atom, R^{N1} and R^{N4} are alkyl groups having 1 to 10 carbon atoms which optionally have a hydroxy group and optionally include one or more oxygen atoms in the chain, and R^{N2} is preferably absent.

The triazolium cation represented by the formula (N1) is preferably a triazolium cation represented by any one of the formulas (1-1) to (1-3).

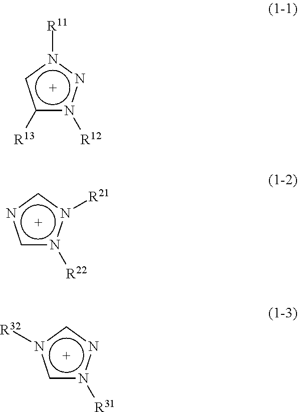

In formula (1-1), R¹¹ and R¹² are alkyl groups having 1 to 10 carbon atoms which optionally have a hydroxy group and optionally include one or more oxygen atoms in the chain; R¹³ is a hydrogen atom or an alkyl group having 1 to 10 carbon atoms which optionally have a hydroxy group and optionally include one or more oxygen atoms in the chain; R¹, R¹² and R¹³ are the same or different from each other;
in formula (1-2), R²¹ and R²² are alkyl groups having 1 to 10 carbon atoms, which optionally have a hydroxy group and optionally include one or more oxygen atoms in the chain; R²¹ and R²² are the same or different from each other; and
in formula (1-3), R³¹ and R³² are alkyl groups having 1 to 10 carbon atoms, which optionally have a hydroxy group and optionally include one or more oxygen atoms in the chain; R³¹ and R³² are the same or different from each other.

Regarding R^{N1}, R^{N2}, R^{N3}, R^{N4}, R^{N5}, R¹, R¹², R¹³, R²¹, R²², R³¹ and R³² (hereinafter, these groups may be simply written as R, which is the general name), R optionally has a hydroxy group, and optionally includes one or more oxygen atoms in the chain of an alkyl group having 1 to 10 carbon atoms as described above. The expression that optionally including one or more oxygen atoms in the chain means that the alkyl group is optionally-(alkyl chain-0)-alkyl group (n is 0 or a positive integer, preferably an integer of 0 to 2). In other words, the end of the alkyl chain is not an oxygen atom. On this occasion, each substituent optionally has a hydroxy group. For example, the alkyl group at an end in the formula is optionally accompanied by a hydroxy group. Alternatively, the intermediate alkyl chain (alkylene group) optionally has a hydroxy group at an optional place.

Regarding R, in the case where the specific alkyl group is an -(alkyl chain-O)ₙ-alkyl group and n is 1, an alkoxy alkyl group having 2 to 10 carbon atoms is preferred, and an alkyl group having 1 to 4 carbon atoms, substituted with an alkoxy group having 1 to 3 carbon atoms is more preferred.

In the present embodiment, it is preferable that R be each independently a hydrogen atom, —(CH₂)ₙ₁CH₃ (n1 is an integer of 0 to 9), and —(CH₂)ₙ₂O(CH₂)ₙ₃CH₃ (n2 is an integer of 1 to 4, and n3 is an integer of 0 to 2).

In each formula, at least one R is the specific alkyl group. Optionally, the substituent R further has a substituent (for example, a hydroxy group, a sulfanyl group (thiol group), etc.) as long as the performance is not greatly impaired. Alternatively, triazole optionally has the substituent and the specific alkyl group, etc., on the carbon atom of its mother nucleus in the range where the performance is not greatly impaired.

It is preferable that the triazolium cation for use in the present embodiment have a molecular weight of 110 to 500.

Specific examples of the triazolium cation for use in the present embodiment are shown in the following, though the present dislosure is not limited thereto without mentioning.

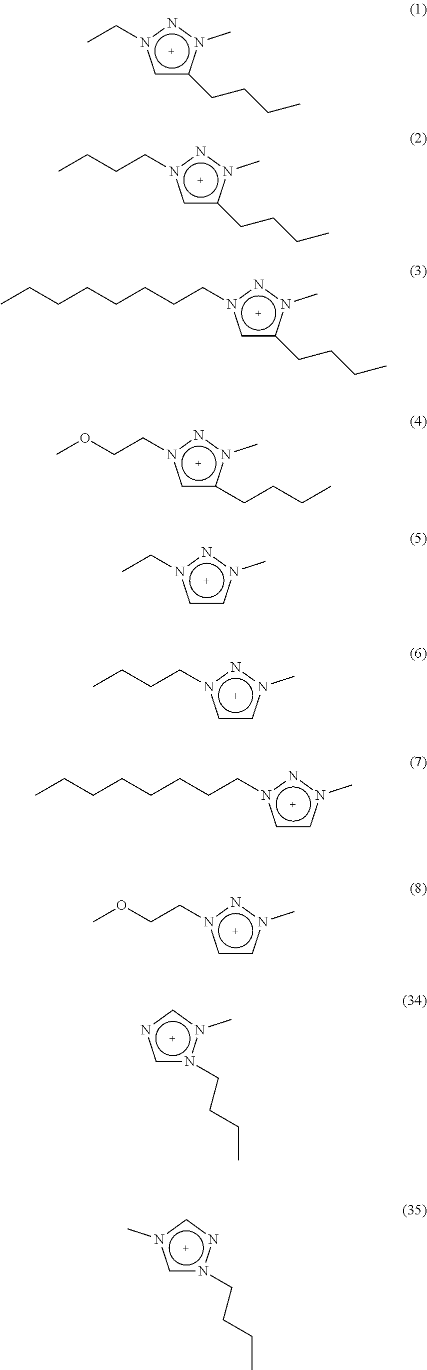

### <Phosphate Ester Anion>

The types and the like of the phosphate ester anion for use in the present embodiment are not particularly specified as long as a salt can be formed with the triazolium cation represented by the formula (N1).

In the present embodiment, it is preferable that the phosphate ester anion be an anion represented by a formula (N2). Use of such a phosphate ester anion allows a stable salt to be formed with the triazolium cation represented by the formula (N1), so that generation of odor can be effectively suppressed. Incidentally, the term phosphate ester anion in the present dislosure includes, an anion of phosphonate (phosphite) ester and an anion of phosphinate (hypophosphite) ester in addition to an anion of phosphate ester.

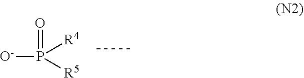

In the formula (N2), R⁴ is a hydrogen atom, a hydroxyl group, an alkyl group having 1 to 6 carbon atoms, a hydroxyalkyl group having 1 to 6 carbon atoms, an alkoxy group having 1 to 6 carbon atoms, an alkenyl group having 2 to 6 carbon atoms, a poly(alkyleneoxy) group having 10 or less carbon atoms, an alkylthio group having 1 to 6 carbon atoms, or a poly(alkylene thio) group having 10 or less carbon atoms, and R⁵ is a hydrogen atom, an alkyl group having 1 to 6 carbon atoms, an alkoxy group having 1 to 6 carbon atoms, an alkenyl group having 2 to 6 carbon atoms, a poly(alkyleneoxy) group having 10 or less carbon atoms, an alkylthio group having 1 to 6 carbon atoms, or a poly(alkylene thio) group having 10 or less carbon atoms.

In the formula (N2), R⁴ is a hydrogen atom, a hydroxyl group, an alkyl group having 1 to 6 carbon atoms, a hydroxy alkyl group having 1 to 6 carbon atoms, an alkoxy group having 1 to 6 carbon atoms, an alkenyl group having 2 to 6 carbon atoms, a poly(alkyleneoxy) group having 10 or less carbon atoms, an alkylthio group having 1 to 6 carbon atoms, or a poly(alkylene thio) group having 10 or less carbon atoms. An alkyl group having 1 to 6 carbon atoms and an alkoxy group having 1 to 6 carbon atoms are preferred, an alkoxy group having 1 to 6 carbon atoms is more preferred, an alkoxy group having 1 to 3 carbon atoms is still more preferred, and a methoxy group and an ethoxy group are furthermore preferred.

In the formula (N2), R⁵ is a hydrogen atom, an alkyl group having 1 to 6 carbon atoms, an alkoxy group having 1 to 6 carbon atoms, an alkenyl group having 2 to 6 carbon atoms, a poly(alkyleneoxy) group having 10 or less carbon atoms, an alkylthio group having 1 to 6 carbon atoms, or a poly(alkylene thio) group having 10 or less carbon atoms. An alkyl group having 1 to 6 carbon atoms and an alkoxy group having 1 to 6 carbon atoms are preferred, an alkoxy group having 1 to 6 carbon atoms is more preferred, an alkoxy group having 1 to 3 carbon atoms is still more preferred, and a methoxy group and an ethoxy group are furthermore preferred.

It is preferable that the phosphate ester for use in the present embodiment have a molecular weight of 64 to 650.

Specific examples of the phosphate ester anion for use in the present embodiment are shown in the following. The phosphate ester anions in the present embodiment are not limited thereto without mentioning.

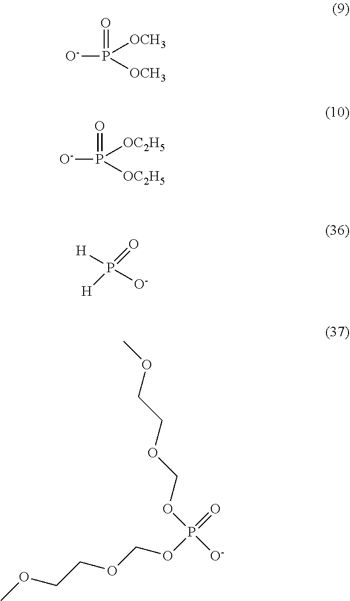

### <Triazolium Cationic Salt>

The triazolium=alkyl phosphate as specific salt is represented by a formula (N31), and specifically a salt represented by a formula (31), (32) or (33) is preferred.

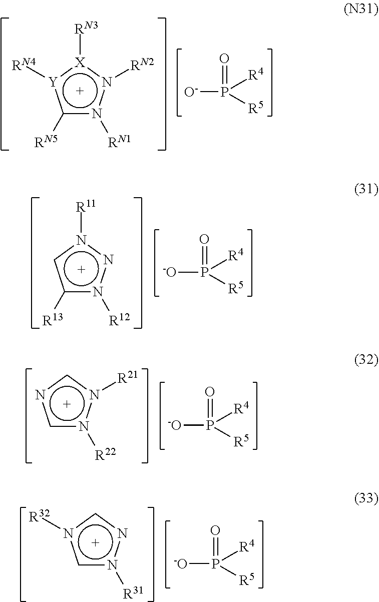

In the formula, R (R^{N1}, R^{N2}, R^{N3}, RNA, R^{N5}, R¹, R¹², R¹³, R²¹, R²², R³¹ and R³²) are the same as the respective groups (R) in the formulas (N1), (1-1), (1-2) and (1-3), and the preferred ranges are also the same.

Typical examples of the salt composed of the triazolium cations and the dimethyl phosphate or diethyl phosphate anions include the following formulas (11) to (18), (36) and (37).

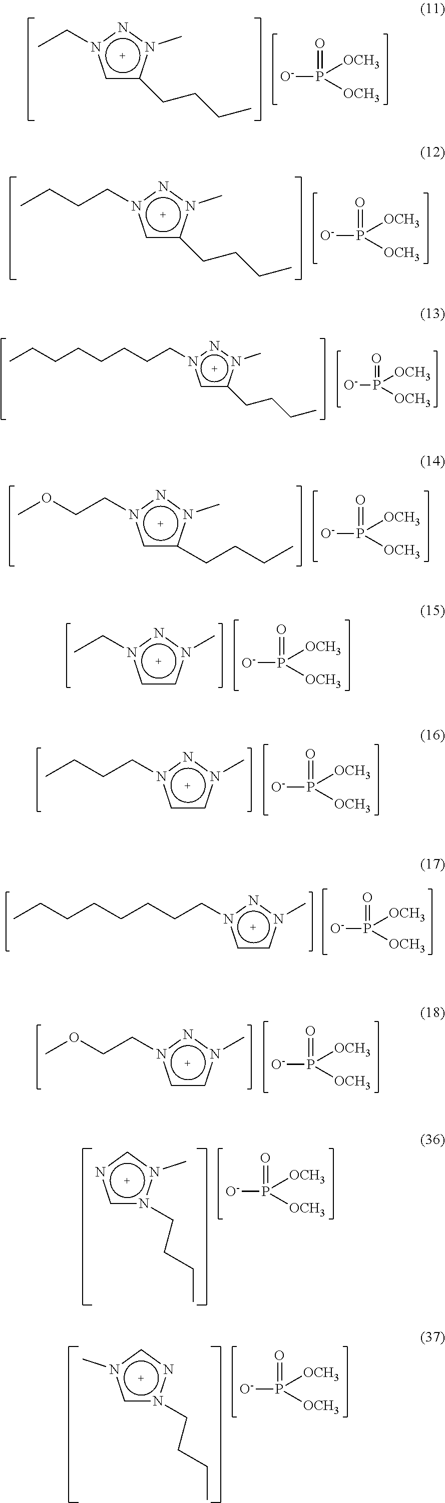

wherein Me represents a methyl group.

In the case of the formula (N1), a triazole having a structure of the triazolium with any one of R^{N1}, R^{N2}, R^{N3} and R^{N4} excluded is reacted with a trialkyl phosphate to make an onium compound, so that a specific salt can be synthesized. The synthesizing process allows the reaction to proceed easily, so that a specific salt can be synthesized in a very simple manner. Further, a metal exchange reaction or an anion exchange with use of an ion exchange resin is not required, so that contamination with a halogen that causes corrosion can be suppressed.

Incidentally, the specific salt may be a solid at normal temperature, or may be a liquid (so-called normal-temperature molten salt (ionic liquid)).

### <Moisture Control Material>

The moisture control material may contain one type of specific salt, or may contain two or more specific salts with one or both of the cation and the anion being different.

The moisture control material may further contain other components usually used in moisture control materials in the range where the effect of the present embodiment is not impaired.

The moisture control material may be an aqueous solution. In this case, the specific salt is contained in the cationic and anionic states.

The moisture control material in the present embodiment may be applied to, for example, a desiccant-type air conditioner and an absorption refrigerator. The moisture control material in the present embodiment may be used in any aspect of an open system and a closed system. In the case of use in an aspect of an open system, the moisture control material including the phosphate ester anion that constitutes the specific salt is particularly suitable from the view point of suppression of odor.

In another embodiment, the salt is preferably an ionic liquid. The ionic liquid means one having a melting point of 100° C. or less at one atmospheric pressure. In particular, the moisture control material in the present embodiment is preferably in a liquid state at least in the range of 0 to 5° C.

The moisture control material in the present embodiment may be an aqueous solution as described above, and is preferably an aqueous solution. In this case, as described above, the specific salt is contained in the cationic and anionic states. The amount of water is preferably 10 mass % or more, more preferably 15 mass % or more, relative to the total amount of the salt (ionic liquid) and water. Also, the amount of water is preferably 95 mass % or less, more preferably 80 mass % or less, and particularly preferably 60 mass % or less.

Further, the moisture control material in the present embodiment may further contain other components usually used in moisture control materials in the range where the effect of the present dislosure is not impaired.

The moisture control material in the present embodiment may be used for an apparatus such as an air conditioner and an absorption refrigerator. As examples of such devices, the descriptions in Japanese Patent Laid-Open No. 2006-142121, Japanese Patent Laid-Open No. 2018-144029, Japanese Patent Laid-Open No. 2020-30004, etc. may be referred to. In particular, since the generation of odor is suppressed, use in open systems such as a desiccant-type air conditioner is particularly suitable.

The moisture control material in the present embodiment may be suitably applied to, for example, a desiccant-type air conditioner and an absorption refrigerator. The moisture control material in the present embodiment may be used in any aspect of an open system and a closed system. In the case of use in an aspect of an open system, the moisture control material including the phosphate ester anion that constitutes the specific salt is particularly suitable from the view point of suppression of odor.

The triazolium phosphate solution that constitutes the moisture control material in the present embodiment preferably has low viscosity. The viscosity of the aqueous solution (for example, 70 mass % aqueous solution) at 25° C. is preferably 75 mPa·s or less, more preferably 50 mPa·s or less, and still more preferably 30 mPa·s or less. With a low viscosity of the triazolium phosphate, it is expected that the transfer efficiency of the moisture control material aqueous solution and the water vapor exchange efficiency resulting from contact with ambient air can be improved. The lower limit is practically 10 mPa·s or more, though not limited thereto.

The moisture control material in the present embodiment has low corrosiveness to various metals such as zinc, copper, aluminum and stainless steel.

According to the constitution, a moisture control material having a high moisture absorption coefficient can be obtained. Further, since the generation of odor can be suppressed, use in open systems such as a desiccant-type air conditioner is particularly suitable.

### EXAMPLES

The present dislosure will be described more specifically with reference to Examples as follows. The materials, amounts used, ratios, processing details, processing procedures, etc. shown in the following Examples may be changed appropriately without departing from the gist of the present dislosure. Accordingly, the scope of the present dislosure is not limited to the following specific examples.

In the case where the measuring instruments and the like used in Examples are hardly available due to discontinued production or the like, other instruments having equivalent performance may be used for the measurement.

Unless otherwise specified, in the present Example, the environment was at 23° C. and a relative humidity of 40%.

### <Synthesis of Triazolium Salt>

### Example 1: Synthesis of 4-butyl-1-ethyl-3-methyl-1,2,3-triazolium=dimethyl phosphate (11)

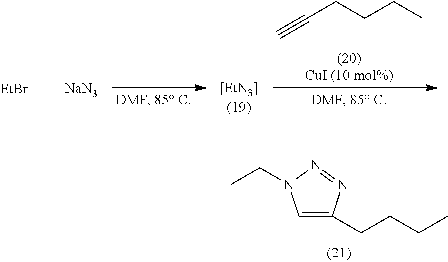

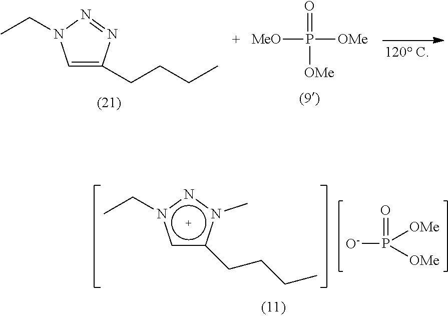

In a 1-L three-neck eggplant flask equipped with a Dimroth condenser and a 100-ML cylindrical dropping funnel, sodium azide (NaN₃) (26.0 g, 400 mmol) was weighed out and 50-mL of DMF was added thereto at room temperature after purging with argon (incompletely dissolved). At room temperature, a DMF solution (25 mL) of ethyl bromide (21.8 g, 200 mmol) was dropped thereto over 30 minutes. After completion of the dropping, the mixture was stirred at 80° C. for 24 hours, so that a white precipitation was deposited in the reaction system. After cooling to room temperature, copper iodide (CuI) (3.81 g, 20 mmol) was fed thereto through a side pipe, so that the solution turned orange color. Subsequently, a DMF solution (25 mL) of 1-hexine (20) (19.7 g, 240 mmol) was dropped through the dropping funnel over 30 minutes, and the mixture was stirred at 80° C. for 24 hours. Incidentally, along with dropping of 1-hexine (20), the orange color faded away and a greyish white suspension was formed. After confirming that the reaction liquid was cooled to room temperature, dilution with ethyl acetate (100 mL) was performed. Copper iodide was removed through celite filtration. After filtration, the filtrate was colored into a dark green. From the filtrate, DMF was removed by a rotary evaporator (70° C.). The residue was dissolved in ethyl acetate (100 mL) and diethyl ether (20 mL), and then washed with water (three times). The organic layer was dried over anhydrous MgSO₄, and through a rotary evaporator and subsequent vacuum drying, a brown liquid (42.36 g) was obtained. The liquid was subjected to Claisen distillation under a reduced pressure (4.8 hPa), so that a colorless liquid of 1-ethyl-4-butyl-1,2,3-triazole (21) (26.2 g, 171 mmol, bp: 97° C./4.6 hPa) was obtained at a yield of 85%.

In a 500-mL one-neck eggplant flask, triazole (21) (26.2 g, 171 mmol) was weighed out, and trimethyl phosphate (9) (205 mmol, 28.70 g) was added directly thereto at room temperature. The mixture was stirred in an argon atmosphere at 120° C. for 24 hours. After cooling to room temperature, decantation cleaning with hexane was performed (3 times). Then, 100 mL of methanol was added for dissolution, and activated charcoal (5.0 g) was added and stirred at 50° C. for 1 hour. After removal of the activated charcoal through celite filtration, vacuum drying (65° C., 5.5 hPa) was performed for 5 hours, so that a pale brown liquid of 4-butyl-1-ethyl-3-methyl-1,2,3-triazolium=dimethyl phosphate (11) (45.74 g, 156 mmol) was obtained at a yield of 91%. The salt of triazolium ion is a liquid at room temperature, and the aqueous solution thereof exhibits neutral.

¹H NMR (500 MHZ, ppm, CDCl₃-DMSO-d₆) δ 0.98 (3H, t, J=7.3 Hz), 1.47-1.49 (2H, m), 1.67 (3H, t, J=6.7 Hz), 1.75-1.85 (2H, m), 2.89 (2H, t, J=7.8 Hz), 3.58 (6H, s), 4.22 (3H, s), 4.83 (2H, q, J=7.1 Hz), 9.70 (1H, s); ¹³C NMR (125 MHz, ppm, CDCl₃-DMSO-d₆) δ 13.6, 14.7, 22.2, 23.1, 29.1, 37.6, 49.3, 52.5, 130.3, 144.3; ESI-MS m/z (M⁺) calcd for C₉H₁₈N₃⁺ 168.1502, found 168.1486, m/z (X⁻) calcd for C₂H₆O₄P⁻ 125.0004, found 124.9990.

The structures and chemical formula numbers of triazolium salts as specific salts that were synthesized by the same method are represented by the compounds (12), (13) and (14).

### Example 2: Synthesis of 1-butyl-3-methyl-1, 2, 3-triazolium=dimethyl phosphate (16)

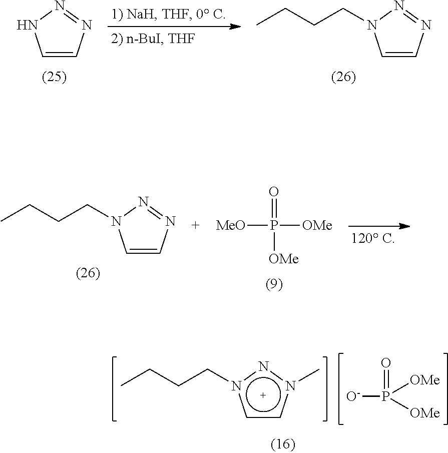

In a 500-mL three-neck flask equipped with a Dimroth condenser and a cylindrical dropping funnel (100 mL), sodium hydride (NaH, ca. 60% in mineral oil) (4.00 g, 100 mmol) was weighed out. After purging with argon, dry hexane was added thereto at room temperature to produce a suspension. The supernatant of the suspension was taken out with a syringe two times, so that the mineral oil for covering was removed. Subsequently, 50 mL of dry THF was added thereto. Into the resulting suspension, a dry THE solution (25 mL) of 1H-1, 2, 3-triazole (25) (6.91 g, 100 mmol) was dropped at 0° C. over 40 minutes. Then the mixture was stirred at room temperature, for 1 hour. Along with the dropping, hydrogen gas was generated, and a greyish white suspension was obtained. Into the suspension, a THE solution (25 mL) of 1-butane iodide (20.2 g, 110 mmol) was dropped over 30 minutes. After completion of the dropping, through stirring in an argon atmosphere at 70° C. for 48 hours, the greyish white suspension was changed into a white suspension. After cooling to room temperature, water (1.80 g, 100 mmol) at 0° C. was added to decompose excessive sodium hydride, so that a transparent pale brown solution was obtained. Anhydrous sodium sulfate was added thereto and stirred for dehydration. The filtrate was concentrated with an evaporator, and then subjected to silica gel flash column chromatography, so that (hexane-ethyl acetate 10:1 to 1:2) 1-butyl-1H-1, 2, 3-triazole (26) (9.45 g, 75.5 mmol) was obtained at a yield of 76%.

In a 300-mL one-neck eggplant flask, triazole (26) (9.45 g, 75.5 mmol) was weighed out, and trimethyl phosphate (9) (11.63 g, 83.0 mmol) was added directly thereto at room temperature. The mixture was stirred in an argon atmosphere at 120° C. for 48 hours. After cooling to room temperature, decantation cleaning with hexane was performed (3 times). Then, 100 mL of ethyl acetate was added for dissolution, and activated charcoal (5.0 g) was added and stirred at 40° C. for 1 hour. After removal of the activated charcoal through celite filtration, the filtrate was concentrated with a rotary evaporator and subjected to vacuum drying, so that 1-butyl-3-methyl-1, 2, 3-triazolium=dimethyl phosphate (16) (16.47 g, 62.1 mmol) was obtained at a yield of 83%. ¹H NMR (80 MHZ, ppm, CDCl₃) δ 1.07 (3H, t, J=6.4 Hz), 1.25-1.73 (2H, m), 1.92-2.08 (2H, m), 3.61 (3H, s), 3.74 (3H, s), 4.58 (3H, s), 4.84 (2H, t, J=6.4 Hz), 9.90 (2H, s); ¹³C NMR (20 MHz, ppm, CDCl₃) δ 13.4, 19.5, 31.7, 40.1, 52.4, 52.7, 53.6, 132.5, 133.3; HRMS (ESI) C₇H₁₄N₃⁺ 140.11883; found 140.1189; HRMS (ESI) calcd for C₂H₆O₄P⁻ 125.000382; found 124.9995.

The structures and chemical formula numbers of triazolium salts as specific salts that were synthesized by the same method are represented by the exemplified compounds (15), (17) and (18).

### <Moisture Absorption Test, Example 3>

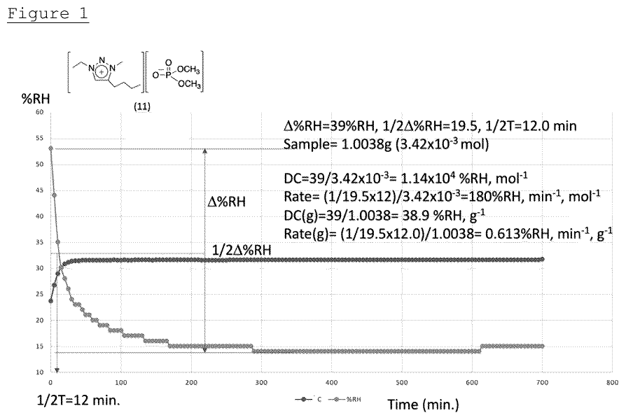

A petri dish with 1.0038 g of the specific salt (11) in Example 1 dispensed therein was placed in a zippered plastic bag having an internal capacity of 1,110 cm³ (manufactured by Asahi Kasei Home Products Corporation, Ziploc (registered trademark), 273 mm by 268 mm) together with a hygrometer (TR-74Ui, illuminance/ultraviolet/temperature/humidity data logger manufactured by T&D Corporation), and the plastic bag was sealed. This was placed in a constant-temperature vessel at 30° C. and allowed to stand still. The change in humidity inside the plastic bag was measured until the humidity inside the plastic bag reached an equilibrium state. The results are shown in graph in FIG. 1. As shown in FIG. 1, the humidity decreases over time, indicating that the specific salt (11) has moisture absorption properties.

In the same method, 1.000 g of each of the specific salts (12), (13), (14), (15) and (16) was placed in a petri dish and subjected to the same moisture absorption test as for the specific salt (11). The results are shown in Table 1.

Comparison of moisture absorption performances of the typical specific salts (11), (12), (13), (14), (15) and (16), two types of commercially available desiccants (silica gel and lithium chloride), [HMC6] [DMPO4]2 described in Non Patent Document 9, and a 30 mass % lithium chloride aqueous solution currently used in the moisture control material.

TABLE 1

| | | Moisture | Moisture | Moisture | Moisture |
|---|---|---|---|---|---|
| | | absorption | absorption | absorption | absorption |
| | | capacity | rate per | coefficient | rate per |
| | Moisture control | per mole | mole | per gram | gram |
| | material | (%/mol) | (%/min, mol) | (%/g) | (%/min, g) |
| Example 3-1 | Specific salt (11) | 11400 | 180 | 38.9 | 0.613 |
| Example 3-2 | Specific salt (12) | 10900 | 477 | 33.9 | 1.35 |
| Example 3-3 | Specific salt (13) | 13500 | 740 | 35.9 | 1.96 |
| Example 3-4 | Specific salt (14) | 17500 | 690 | 29.4 | 1.18 |
| Example 3-5 | Specific salt (15) | 7110 | 418 | 30.0 | 1.76 |
| Example 3-6 | Specific salt (16) | 6850 | 439 | 25.8 | 1.66 |
| Comparative | SiO₂ (Silica gel) | 1710 | 687 | 28.4 | 1.56 |
| Example 3-1^{[a]} | (FUJIFILM | | | | |
| | Wako, Lot No. | | | | |
| | CTH2255) | | | | |
| Comparative | Lithium chloride | 1350 | 894 | 32.0 | 2.1 |
| Example 3-2^{[a]} | (FUJIFILM | | | | |
| | Wako, Lot No. | | | | |
| | HPQ6248) | | | | |
| Comparative | [HMC6][DMPO₄]₂ | 20000 | 900 | 44.0 | 1.9 |
| Example 3-3^{[a]} | | | | | |
| Comparative | 30 mass % | 56 | 4.68 | 1.32 | 0.11 |
| Example 3-4^{[a]} | lithium chloride | | | | |
| | aqueous | | | | |
| | solution | | | | |

^{[a]}ldescribed in Non Patent Document 9

In comparison with Comparative Example 3-1 with use of silica gel for drying and Comparative Example 3-2 with use of lithium chloride shown in Table 1, the specific salt (11) to the specific salt (16) had higher moisture absorption rate. However, the moisture absorption coefficient and the moisture absorption rate were inferior to N¹, N¹, N¹, N⁶, N⁶, N⁶-hexamethyl hexane-1,6-diaminium=dimethyl phosphate ([HMC6] [DMPO₄]₂) described in Non Patent Document 9 shown in Comparative Example 3-3.

The specific salt (11) had a moisture absorbency 7 times per mole, or 1.4 times per gram, higher than the silica gel that is currently widely used as moisture control material (Table 1, Comparative Example 3-1). Among the specific salt (11) to the specific salt (16), the specific salt (14) had the highest moisture absorbency, i.e. 10 times higher per mole than silica gel (Table 1, Comparative Example 3-1), though having an equivalent moisture absorbency per gram.

It has been found that the moisture absorption coefficients per mole of the specific salts (12) to (16) are 4 times to 10 times higher per mole in comparison with the silica gel that is currently widely used as moisture control material (Table 1, Comparative Example 3-1). The moisture absorption properties per mole of the specific salts (11) to (16) were in the order of (14)>(13)>(11)>(12)>(15)>(16), and the moisture absorption properties per gram were in the order of (11)>(13)>(12)>(15)>(14)>(16). Although there has been reported that an ionic liquid having a smaller cation portion has higher moisture absorption properties (Non Patent Document 3), it has been found that the rule does not apply to the specific salts (11) to (16) as ionic liquids of triazolium salt.

### <Measurement of Equilibrium Water Vapor Pressure, Example 4>

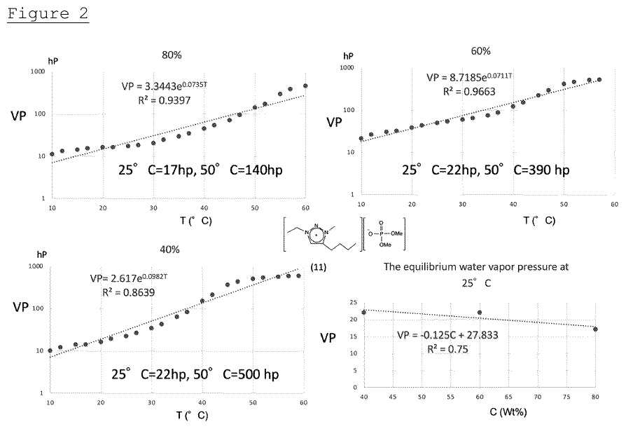

Aqueous solutions of 80 mass %, 60 mass % and 40 mass % specific salt (11) synthesized in Example 1 were prepared to measure the equilibrium water vapor pressure at each temperature ranging from 20° C. to 60° C. The results are shown in FIG. 2.

Aqueous solutions of 80 mass %, 60 mass % and 40 mass % specific salts (12), (13), (14), (15) and (16) were prepared by the same method to measure the equilibrium water vapor pressure at each temperature ranging from 20° C. to 60° C. The equilibrium water vapor pressures of the specific salts (11) to (16) at 25° C. and 50° C. are shown in Table 2.

Equilibrium water vapor pressures of the specific salts (11) to (16) at 25° C. and 50° C.

TABLE 2

| | | Aqueous | Equilibrium | Equilibrium |
|---|---|---|---|---|
| | | solution | water vapor | water vapor |
| | | concentration | pressure | pressure |
| | Specific salt | (mass %) | (25° C.) | (50° C.) |
| Example 4-1 | Specific salt (11) | 80 | 17 hPa | 123 hPa |
| Example 4-2 | Specific salt (11) | 60 | 22 hPa | 390 hPa |
| Example 4-3 | Specific salt (11) | 40 | 22 hPa | 500 hPa |
| Example 4-4 | Specific salt (12) | 80 | 17 hPa | 150 hPa |
| Example 4-5 | Specific salt (12) | 60 | 27 hPa | 410 hPa |
| Example 4-6 | Specific salt (12) | 40 | 32 hPa | 450 hPa |
| Example 4-7 | Specific salt (13) | 80 | 21 hPa | 150 hPa |
| Example 4-8 | Specific salt (13) | 60 | 31 hPa | 440 hPa |
| Example 4-9 | Specific salt (13) | 40 | 37 hPa | 540 hPa |
| Example 4-10 | Specific salt (14) | 80 | 27 hPa | 260 hPa |
| Example 4-11 | Specific salt (14) | 60 | 24 hPa | 420 hPa |
| Example 4-12 | Specific salt (14) | 40 | 23 hPa | 510 hPa |
| Example 4-13 | Specific salt (15) | 80 | 29 hPa | 330 hPa |
| Example 4-14 | Specific salt (15) | 60 | 27 hPa | 380 hPa |
| Example 4-15 | Specific salt (15) | 40 | 28 hPa | 450 hPa |
| Example 4-16 | Specific salt (16) | 80 | 25 hPa | 280 hPa |
| Example 4-17 | Specific salt (16) | 60 | 27 hPa | 470 hPa |
| Example 4-18 | Specific salt (16) | 40 | 29 hPa | 510 hPa |
| Comparative | [P_{1,4,4,4}][DMPO₄] | 77 | 21 hPa | 91 hPa |
| Example 4-1^{[a]} | | | | |
| Comparative | [HMC6][DMPO₄]₂ | 80 | 23 hPa | 140 hPa |
| Example 4-2^{[b]} | | | | |
| Comparative | Lithium chloride | 30 | 24 hPa | 150 hPa |
| Example 4-3^{[b]} | | | | |

^{[a]}described in Non Patent Document 7,
^{[b]}described in Non Patent Document 9

As shown in Example 4-1 in Table 2, the 80 mass& specific salt (11) aqueous solution had a very low equilibrium water vapor pressure of 17 hPa at 25° C., and the 40 mass % aqueous solution also had a low equilibrium water vapor pressure of 22 hPa at 25° C. Further, it has been found that the 40 mass % specific salts (11) and (14) aqueous solutions also had equilibrium water vapor pressures at 25° C. somewhat lower in comparison with the 30 mass % lithium chloride aqueous solution shown in Comparative Example 4-3 in Table 2.

Since a moisture control material for a liquid-type desiccant air conditioner is required to smoothly absorb and desorb water vapor from and to the ambient air to be contacted, it is desired to have a low equilibrium water vapor pressure at low temperature and a high equilibrium water vapor pressure at high temperature. From such a viewpoint, the equilibrium water vapor pressure at 25° C. is preferably 100 hPa or less, more preferably 70 hPa or less, and still more preferably 50 hPa or less. The lower limit is practically 10 hPa or more, though not limited. The equilibrium water vapor pressure at 50° C. is preferably 150 hPa or more, more preferably 200 hPa or more, still more preferably 250 hPa or more, and furthermore preferably 300 hPa or more. The upper limit is practically 800 hPa or less, though not limited.

From the results shown in Examples 4-1 and 4-2 in Table 2, it has been shown that the features of the 80 mass % and 60 mass % specific salt (11) aqueous solutions as moisture control material are more excellent than those of a 30 mass % lithium chloride aqueous solution. The equilibrium water vapor pressure of the 40 mass % specific salt (11) aqueous solution shown in Example 4-3 in Table 2 is equivalent to that of the 30 mass % lithium chloride aqueous solution, and at 50° C., the equilibrium water vapor pressure is higher in comparison with a 30 mass % lithium chloride aqueous solution, which allows smooth water vapor exchange through contact with an ambient air to be achieved.

The experimental results shown in Examples 4-4 to 4-6 in Table 2 show that the features of the 80 mass % specific salt (12) aqueous solution as moisture control material are more excellent than those of the 30 mass % lithium chloride aqueous solution. However, the results show that features of the 60 mass % and 40 mass % specific salt (12) aqueous solutions as moisture control material are inferior to those of the 30 mass % lithium chloride aqueous solution. However, the results show that the moisture control material has a high equilibrium water vapor pressure at 50° C., which allows smooth water vapor exchange through contact with an ambient air to be achieved.

The experimental results shown in Example 4-7 in Table 2 show that the features of the 80 mass % specific salt (13) aqueous solution as moisture control material are equivalent to those of the 30 mass % lithium chloride aqueous solution. On the other hand, although the water vapor absorption features of the 60 mass % and 40 mass % specific salt (13) aqueous solutions as moisture control materials are inferior to those of a 30 mass % lithium chloride aqueous solution, the equilibrium water vapor pressures at 50° C. are high, which allows smooth water vapor exchange through contact with an ambient air to be achieved.

The experimental results shown in Example 4-16 in Table 2 show that the features of the 80 mass % specific salt (16) aqueous solution as moisture control material are slightly inferior or approximately equivalent to those of the 30 mass % lithium chloride aqueous solution. Although the water vapor absorption features of the 60 mass % and 40 mass % specific salt (16) aqueous solutions at 25° C. as moisture control materials are inferior to those of a 30 mass % lithium chloride aqueous solution, the equilibrium water vapor pressures at 50° C. are high, which allows smooth water vapor exchange through contact with an ambient air to be achieved.

### <Corrosive Solubility Test to Metal, Example 5>

It is preferable that the moisture control material for use in an air conditioner have low corrosiveness to the metal of a metal pipe. The 30 mass % lithium chloride aqueous solution currently used as moisture control material has severe corrosiveness to metals such as iron and copper. Accordingly, titanium coating is required for all the piping, resulting in the current slack of liquid-type commercially available air conditioners with costs pushed up, which is a factor of hindering spreading.

Metal pieces (length: 10 mm, width: 15 mm, thickness: 2 mm) made of the following four types of metal materials that are generally used for an air conditioner were prepared.

Fe—Zn: molten zinc-aluminum-magnesium alloy plated steel sheet (trade name: SPHC steel sheet)
Al: corrosion-resistant aluminum A5052
Cu: tough pitch copper C1100P
SUS: stainless steel SUS 304

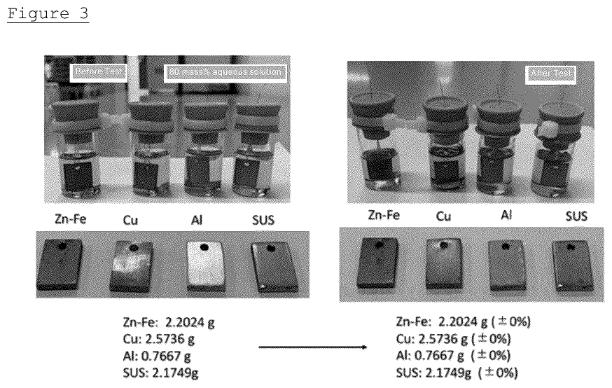

In a sample tube containing 9.0 ml of 80 mass % specific salt (11) aqueous solution in Example 1, a metal piece was placed and held at 80° C. for 2 days. The metal piece was washed with deionized water 5 times and dried under reduced pressure. The mass of the metal piece was measured with an electronic balance before and after the processing to obtain the change in mass of the metal piece before and after the processing. Also, as a comparative test, the 30 mass % lithium chloride and 80 mass % [HMC6] [DMPO₄]₂ aqueous solutions were subjected to the same metal solubility test. The results are shown in FIG. 3 and Table 3. Incidentally, trimethyl phosphate for use as alkylating agent in synthesis of the specific salt (11) has moisture absorption properties and absorbs water during storage to cause hydrolysis, so that phosphoric acid is formed. Even on this occasion, the methylation reaction proceeds smoothly to produce the specific salt (11). However, the resulting specific salt contains a trace amount of phosphoric acid as residual impurities, which may exhibit corrosiveness to metals in some cases. Accordingly, before a corrosion test, checking the acidity of the 80 mass % specific salt aqueous solution is required. When acidity is exhibited, after the specific salt is dissolved in acetone, 5 g of anhydrous potassium carbonate per 30 g of the specific salt (11) is added and stirred, and the mixture is left to stand for 12 hours. Subsequently, anhydrous potassium carbonate is removed through filtration, and acetone is distilled away under reduced pressure, so that phosphoric acid can be removed.

Results of metal solubility test of 80 mass % specific salts (11) and (13) aqueous solutions.

TABLE 3

| | Moisture control | Change in mass (wt %) | | | |
|---|---|---|---|---|---|
| | material | Fe—Zn | Al | Cu | SUS |
| Example 5-1 | 80 mass % | ±0 | ±0 | ±0 | ±0 |
| | Specific salt (11) | | | | |
| | aqueous solution | | | | |
| Example 5-2 | 80 mass % | −0.32 | ±0 | −0.047 | ±0 |
| | Specific salt (13) | | | | |
| | aqueous solution | | | | |
| Comparative | 80 mass % | ±0 | ±0 | −0.035 | ±0 |
| Example | [HMC6][DMPO₄]₂ | | | | |
| 5-1^{[a]} | aqueous solution | | | | |
| Comparative | 30 mass % LiCl | −0.127 | +0.0780 | −0.167 | −0.0324 |
| Example | aqueous solution | | | | |
| 5-2^{[a]} | | | | | |

^{[a]}described in Non Patent Document 9

As shown in Example 5-1 in Table 3, as the results of corrosion experiments with use of the specific salt (11), any of the metals tested had no mass reduction, so that it has been found that the 80 mass % specific salt (11) aqueous solution exhibited no corrosiveness to metals under the present experimental conditions. On the other hand, it has been found that the 80 mass % [HMC6] [DMPO₄]₂ aqueous solution in Comparative Example 5-1 in Table 3 allowed slight corrosion of copper alone, and the 30 mass % lithium chloride aqueous solution in Comparative Example 5-2 in Table 3 caused corrosion of all the metals.

As shown in Example 5-2 in Table 3, as the results of corrosion experiments with use of the specific salt (13), after the corrosion experiment, regarding Fe—Zn, coloring of the aqueous solution occurred, and regarding Fe—Zn and Cu, mass reduction occurred. It has been found that under the present experimental conditions, the specific salt (13) has slight corrosiveness to these metals. However, regarding Al and SUS, no corrosiveness was exhibited at all. Incidentally, it has been observed that the 80 mass % specific salt (13) aqueous solution exhibited neutral by a pH test paper, while exhibiting acidic pH of 4.6 by measurement of acidity using a pH meter. It is presumed that a slight amount of impurities remaining in the specific salt (13) may have affected the electrode of the pH meter. Although a chemical reaction may be caused between the impurities and Fe—Zn, details of the impurities are unidentified.

### <Viscosity Test, Example 6>

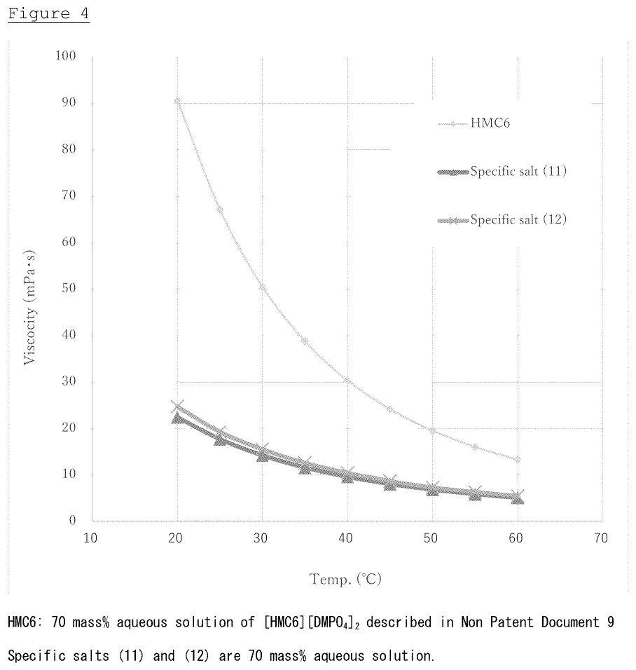

It is desirable that the viscosity of the moisture control material for a liquid-type desiccant air conditioner be as low as possible. As shown in the graph in FIG. 4, the 70 mass % specific salt (11) and specific salt (12) aqueous solutions had lower viscosity in comparison with the 70 mass % [HMC6] [DMPO₄] aqueous solution for use in Comparative Example (Table 1, Comparative Example 3-3, Non Patent Document 9). It has been found that at any temperature, the viscosity of the 70 mass % specific salt (11) or specific salt (12) aqueous solution is lower in comparison with those in Comparative Examples. In a liquid-type desiccant air conditioner, the ambient air is brought into contact with the aqueous solution of moisture control material to perform moisture control of the ambient air and air conditioning temperature control. Further, in order to reduce the resistance of the aqueous solution of moisture control material in the piping and to improve the efficiency of contact with the ambient air, it is preferable that the viscosity of the aqueous solution of moisture control material be low. Although the specific salts (11) and (12) have inferior moisture absorption property in comparison with the 70 mass % [HMC6] [DMPO₄] aqueous solution, the 70 mass % aqueous solutions have low viscosity at each temperature, so that efficient moisture control can be achieved through contact with the ambient air.

### <Equilibrium Water Vapor Pressure, Example 7>

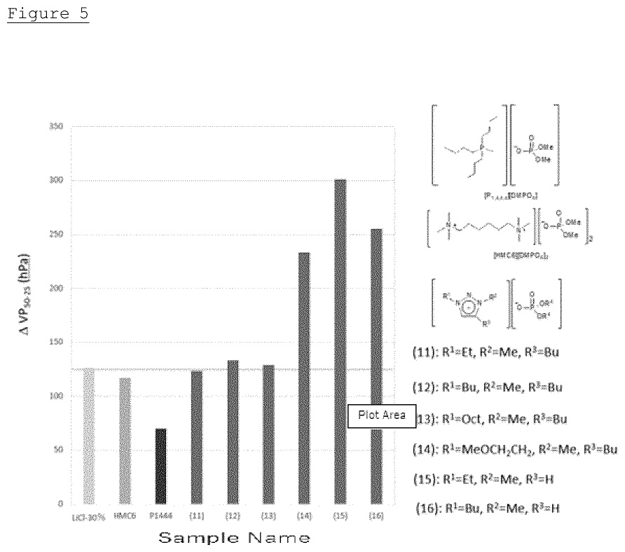

A value of difference between the equilibrium water vapor pressure at 50° C. and the equilibrium water vapor pressure at 25° C. (ΔVP₅₀₋₃₅) of the specific salts (11), (12), (13), (14), (15) and (16) as triazolium salts was calculated to compare with the values of known moisture control materials including a 30 mass % aqueous solution of lithium chloride, a 77 mass % [P_{1, 4, 4, 4}] [DMPO₄] aqueous solution (P1444: Non Patent Document 7), and a 80 mass % [HMC6] [DMPO₄]₂ aqueous solution: Non Patent Document 9). Incidentally, as the equilibrium water vapor pressures of the specific salts (11), (12), (13), (14), (15) and (16), the values of 80 mass % aqueous solution were used. The result graph is shown in FIG. 5. The vertical axis of the graph is the difference in the equilibrium water vapor pressure (unit: hPa).

As shown in FIG. 5, the specific salts (11) to (16) as triazolium salts had a large ΔVP₅₀₋₂₅, and in particular, the specific salts (14), (15) and (16) had a large ΔVP₅₀₋₂₅. Although the moisture absorption capacity per mole and the moisture absorption coefficient per gram of these specific salts (Table 1) are inferior in comparison with [HMC6] [DMPO₄]₂ (Table 1, Comparative Example 3-3), the value of ΔVP₅₀₋₂₅ is large. In particular, the ΔVP₅₀₋₂₅ of the specific salt (15) was about two times the ΔVP₅₀₋₂₅ Of the 30 mass % lithium chloride aqueous solution or the 80 mass % [HMC6] [DMPO₄]₂ aqueous solution. The results indicate that by bringing the aqueous solution of the specific salts (14) to (16) into contact with the ambient air, more efficient water vapor exchange can be achieved in comparison with the known moisture control materials including lithium chloride, [HMC6] [DMPO₄]₂, and [P_{1, 4, 4, 4}] [DMPO₄]. From the viewpoint, ΔVP₅₀₋₂₅ is preferably 50 hPa or more, more preferably 100 hPa or more, and still more preferably 150 hPa or more. The upper limit is practically 500 hPa or less, though not limited.

1. A moisture control material comprising a salt composed of a triazolium cation represented by the following formula (N1) and a phosphate ester anion: 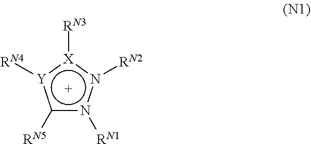
wherein in formula (N1), X and Y represent a nitrogen atom or a methine group, with proviso that any one of X and Y is a nitrogen atom, and another one is a methine group; R^{N1}, R^{N2}, R^{N3}, R^{N4} and R^{N5} are each independently absent, hydrogen atom, an alkyl group having 1 to 10 carbon atoms which optionally has hydroxy group and optionally includes one or more oxygen atoms in the chain; R^{N1}, R^{N2}, R^{N3}, R^{N4} and R^{N5} are the same or different from each other; not all of R^{N1}, R^{N2}, R^{N3}, R^{N4} and R^{N5} are hydrogen atoms or absent; and one or more of R^{N1}, R^{N2}, R^{N3} and R^{N4} substituted on nitrogen atom that constitutes a 5-membered ring are alkyl groups having 1 to 10 carbon atoms, which optionally have hydroxy group and optionally include one or more oxygen atoms in the chain.

2. The moisture control material according to claim 1, wherein in formula (N1), in the case where X is nitrogen atom, R^{N1} and R^{N3} are alkyl groups having 1 to 10 carbon atoms, which optionally have hydroxy group and optionally include one or more oxygen atoms in the chain, and R^{N2} is absent.

3. The moisture control material according to claim 1, wherein in formula (N1), in the case where Y is nitrogen atom, R^{N1} and R^{N2} are alkyl groups having 1 to 10 carbon atoms, which optionally have hydroxy group and optionally include one or more oxygen atoms in the chain, and R^{N4} is absent; alternatively, in the case where Y is nitrogen atom, R^{N1} and R^{N4} are alkyl groups having 1 to 10 carbon atoms, which optionally have hydroxy group and optionally include one or more oxygen atoms in the chain, and R^{N2} is absent.

4. The moisture control material according to claim 1, comprising a salt composed of triazolium cations represented by any one of the following formulas (1-1) to (1-3) and a phosphate ester anion: 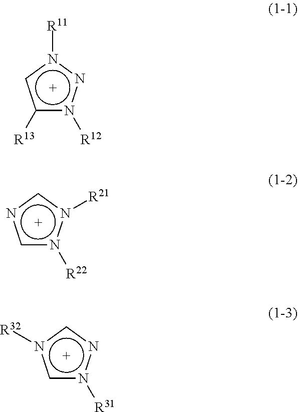
wherein in formula (1-1), R¹¹ and R¹² are alkyl groups having 1 to 10 carbon atoms which optionally have hydroxy group and optionally include one or more oxygen atoms in the chain; R¹³ is hydrogen atom or an alkyl group having 1 to 10 carbon atoms which optionally have hydroxy group and optionally include one or more oxygen atoms in the chain; R¹¹, R¹² and R¹³ are the same or different from each other;
in formula (1-2), R²¹ and R²² are alkyl groups having 1 to 10 carbon atoms, which optionally have hydroxy group and optionally include one or more oxygen atoms in the chain; R²¹ and R²² are the same or different from each other; and
in formula (1-3), R³¹ and R³² are alkyl groups having 1 to 10 carbon atoms, which optionally have hydroxy group and optionally include one or more oxygen atoms in the chain; R³¹ and R³² are the same or different from each other.

5. The moisture control material according to claim 1, wherein the phosphate ester anion comprises an anion represented by formula (N2): 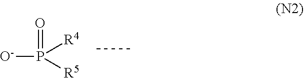
wherein in formula (N2), R⁴ is hydrogen atom, hydroxyl group, an alkyl group having 1 to 6 carbon atoms, a hydroxyalkyl group having 1 to 6 carbon atoms, an alkoxy group having 1 to 6 carbon atoms, an alkenyl group having 2 to 6 carbon atoms, a poly(alkyleneoxy) group having 10 or less carbon atoms, an alkylthio group having 1 to 6 carbon atoms, or a poly(alkylene thio) group having 10 or less carbon atoms; and R⁵ is hydrogen atom, an alkyl group having 1 to 6 carbon atoms, an alkoxy group having 1 to 6 carbon atoms, an alkenyl group having 2 to 6 carbon atoms, a poly(alkyleneoxy) group having 10 or less carbon atoms, an alkylthio group having 1 to 6 carbon atoms, or a poly(alkylene thio) group having 10 or less carbon atoms.

6. The moisture control material according to claim 1, wherein the triazolium cation is represented by any of the following formulas (1) to (8), (34) and (35):

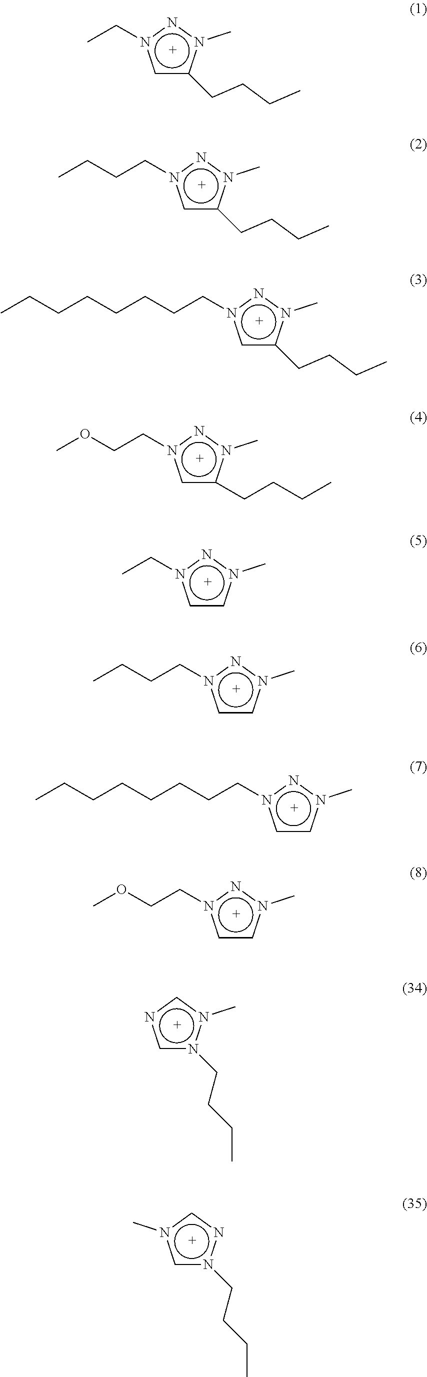

7. The moisture control material according to claim 1, wherein the phosphate ester anion is represented by the following formula (9) or (10):

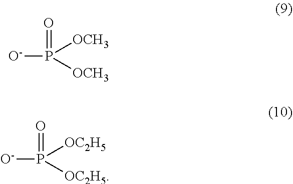 [SMILES: COP(=O)([O-])OC ; CCOP(=O)([O-])OCC]

8. The moisture control material according to claim 1, wherein a solution of the salt composed of triazolium cations and phosphate ester anions has a viscosity of 50 mPa·s or less at 25° C.

9. The moisture control material according to claim 1, having an equilibrium water vapor pressure at 25° C. of 100 hPa or less, and an equilibrium water vapor pressure at 50° C. of 200 hPa or more.

10. The moisture control material according to claim 1, having a value as difference between the equilibrium water vapor pressure at 25° C. and the equilibrium water vapor pressure at 50° C. (ΔVP₅₀₋₂₅) of 50 hPa or more.

11. An apparatus comprising the moisture control material according to claim 1.
